(19) United States
(12) Patent Application Publication
(10) Pub. No.: US 2008/0262566 A1
(43) Pub. Date: Oct. 23, 2008

(54) METHODS AND SYSTEMS OF TREATING MEDICATION OVERUSE HEADACHE

(75) Inventor: Kristen N. Jaax, Santa Clarita, CA (US)

Correspondence Address: ADVANTEDGE LAW GROUP , LLC, 3301 N. UNIVERSITY AVE ., SUITE 200, PROVO, UT, 84604, US

(73) Assignee: Boston Scientific Neuromodulation Corporation, Valencia, CA (US)

(21) Appl. No.: 12/106,970

(22) Filed: Apr. 21, 2008

Related U.S. Application Data
(60) Provisional application No. 60/925,957, filed on Apr. 23, 2007.

(51) Int. Cl. A61N 1/34

(52) U.S. Cl. 607/46

(57) ABSTRACT

Methods and systems of treating a patient with medication overuse headache include providing a stimulator, configuring one or more stimulation parameters to treat medication overuse headache, programming the stimulator with the one or more stimulation parameters, generating a stimulus configured to treat the medication overuse headache with the stimulator in accordance with the one or more stimulation parameters, and applying at least one stimulus with the stimulator to a stimulation site within the patient in accordance with the one or more stimulation parameters.

### RELATED APPLICATIONS

The present application claims priority under 35 U.S.C. §119(e) to U.S. Provisional Patent Application No. 60/925,957 by Kristen N. Jaax et al., filed on Apr. 23, 2007, and entitled “Methods and Systems of Treating Medication Overuse Headache,” the contents of which are hereby incorporated by reference in their entirety.

### BACKGROUND

People who suffer from constant headaches often take a variety of different medications in an effort to ameliorate the effects thereof. For example, preventive medications may be taken in an attempt to prevent the onset of a headache, abortive medications may be taken to stop or “abort” a headache when an aura is sensed, and rescue medications may be taken after a headache is already fully developed.

Unfortunately, many medications have the potential to cause medication overuse headaches (MOH) if used with regularity. In general, medication overuse may be defined in terms of the number of days per month that a particular type of medication is used by a patient to treat headache. For example, the use of various abortive medications (e.g., triptans, opioids, or combination analgesics) for 10 or more days per month is defined as medication overuse by the International Headache Society (IHS). The frequency of use in order to be considered medication overuse may vary depending on the particular medication.

It is not clear if medication overuse causes worsening of headache symptoms or arises as a result of increased headache frequency. Regardless, medication overuse headaches can be debilitating and difficult to overcome. Attempts to wean medication overuse patients off of medications are typically unsuccessful, with a reversion rate of up to seventy percent.

### SUMMARY

Methods of treating a patient with medication overuse headache include providing a stimulator, configuring one or more stimulation parameters to treat medication overuse headache, programming the stimulator with the one or more stimulation parameters, generating a stimulus configured to treat the medication overuse headache with the stimulator in accordance with the one or more stimulation parameters, and applying at least one stimulus with the stimulator to a stimulation site within the patient in accordance with the one or more stimulation parameters.

Systems for treating a patient with medication overuse headache include a stimulator configured to generate at least one stimulus in accordance with one or more stimulation parameters adjusted to treat medication overuse headache, a programmable memory unit in communication with the stimulator and programmed to store the one or more stimulation parameters to at least partially define the stimulus such that the stimulus is configured to treat the medication overuse headache, and means, operably connected to the stimulator, for applying the stimulus to a stimulation site within the patient.

### BRIEF DESCRIPTION OF THE DRAWINGS

The accompanying drawings illustrate various embodiments of the principles described herein and are a part of the specification. The illustrated embodiments are merely examples and do not limit the scope of the disclosure.

FIG. 1A depicts the upper cervical spine area of a patient and shows a number of nerves originating in the upper cervical spine area.

FIG. 1B depicts the occipital nerves in the back of the head and upper neck area of a patient.

FIGS. 1C-1D depict the trigeminal nerve and its branches.

FIG. 2 illustrates an exemplary implantable stimulator according to principles described herein.

FIG. 3 illustrates an exemplary microstimulator according to principles described herein.

FIG. 4A shows an example of a microstimulator with one or more leads coupled thereto according to principles described herein.

FIG. 4B shows an example of a microstimulator with a plurality of electrodes disposed on an outer surface thereof according to principles described herein.

FIG. 4C shows the exemplary microstimulator of FIG. 4B coupled to a lead having a number of electrodes disposed thereon.

FIG. 5 depicts a number of stimulators configured to communicate with each other and/or with one or more external devices according to principles described herein.

FIGS. 6-7 illustrate exemplary configurations wherein one or more electrodes coupled to an implantable stimulator are in communication with one or more of the occipital nerves.

Throughout the drawings, identical reference numbers designate similar, but not necessarily identical, elements.

### DETAILED DESCRIPTION

Methods and systems for treating a patient with medication overuse headache are described herein. A stimulator is configured to apply at least one stimulus a stimulation site within a patient (e.g., one or more of the occipital or trigeminal nerves) in accordance with one or more stimulation parameters. The stimulus is configured to treat medication overuse headache and may include electrical stimulation, drug stimulation, gene infusion, chemical stimulation, thermal stimulation, electromagnetic stimulation, mechanical stimulation, and/or any other suitable stimulation. As used herein, “treating” medication overuse headache refers to any amelioration or prevention of one or more causes, symptoms, and/or sequelae of medication overuse headache. Treating medication overuse headache may additionally or alternatively include facilitating the decrease and/or elimination of medications used in association with medication overuse headache and/or any other medical condition.

In the following description, for purposes of explanation, numerous specific details are set forth in order to provide a thorough understanding of the present systems and methods. It will be apparent, however, to one skilled in the art that the present systems and methods may be practiced without these specific details. Reference in the specification to “one embodiment” or “an embodiment” means that a particular feature, structure, or characteristic described in connection with the embodiment is included in at least one embodiment. The appearance of the phrase “in one embodiment” in various places in the specification are not necessarily all referring to the same embodiment.

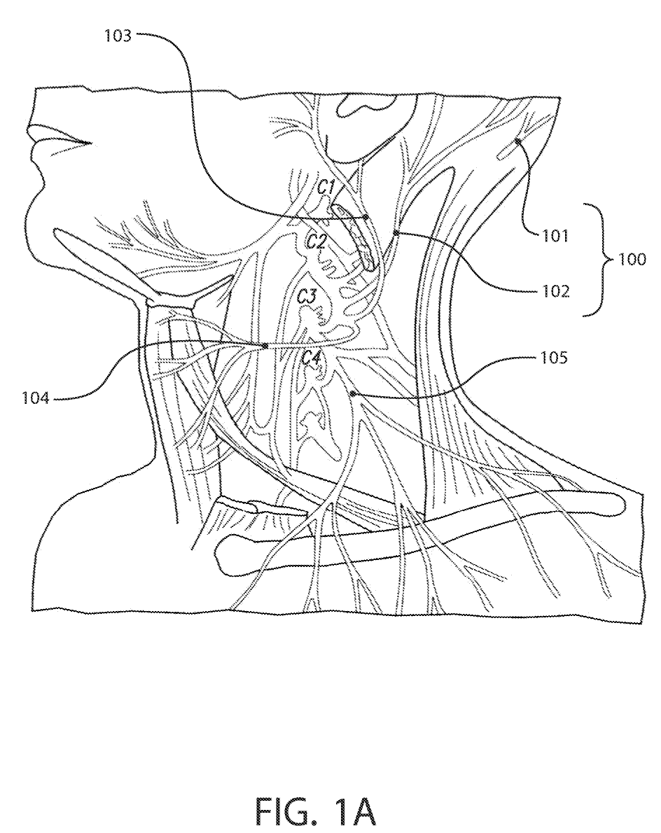

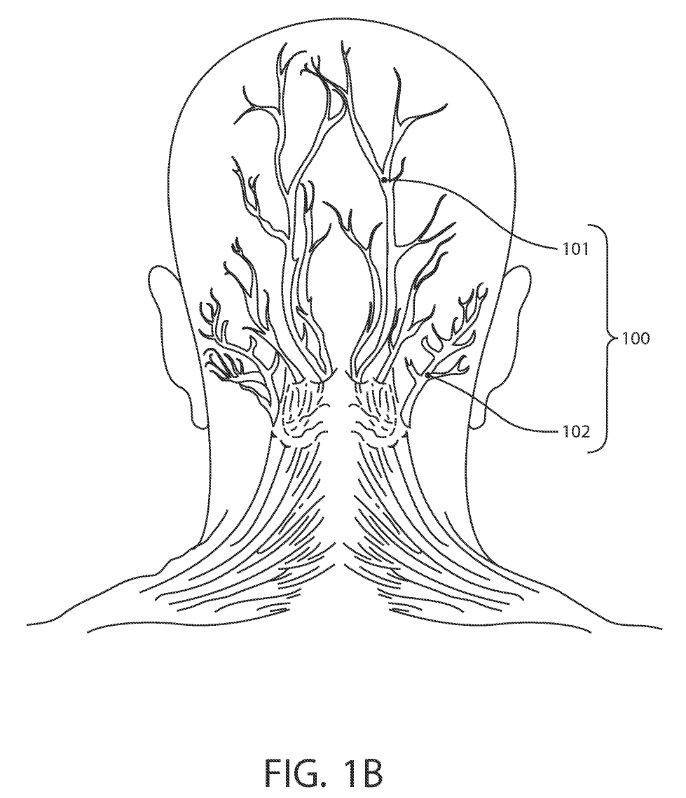

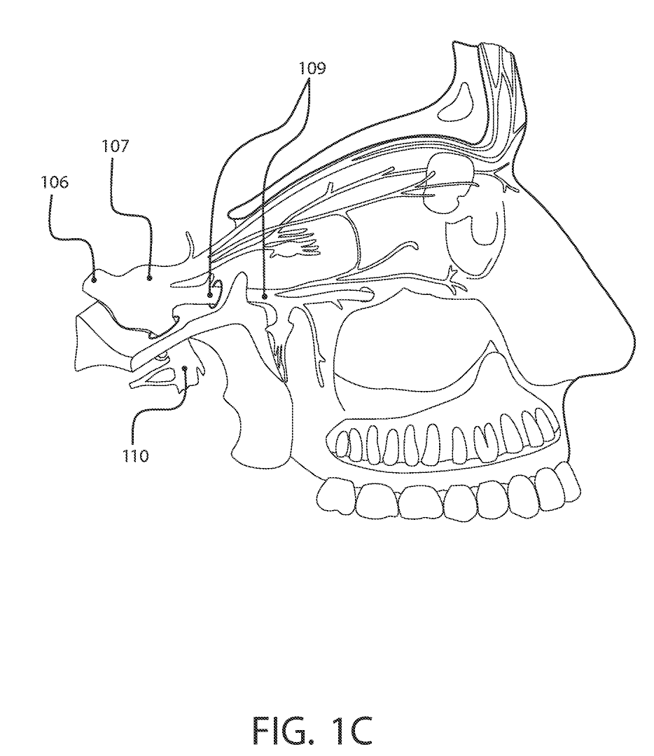

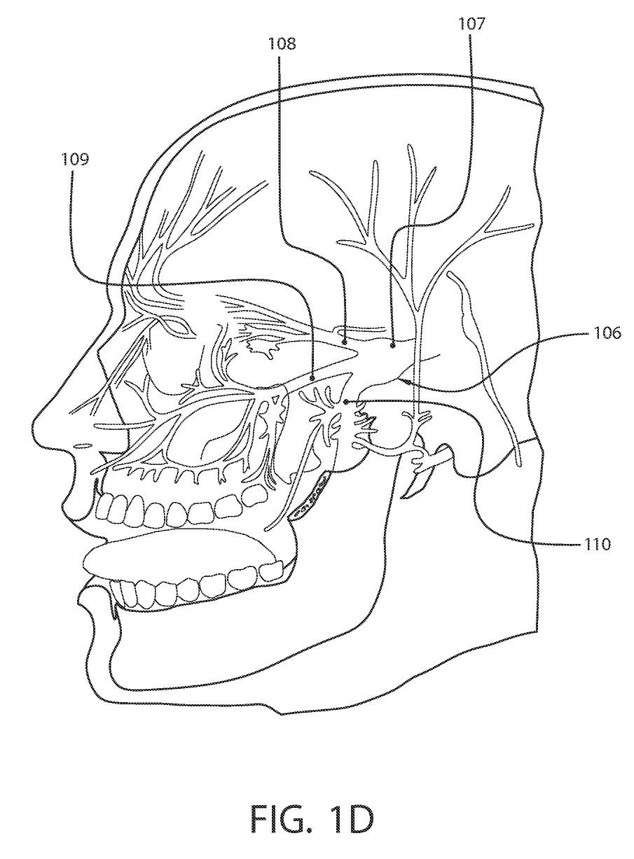

To facilitate an understanding of the systems and methods described herein, a brief overview of the occipital and trigeminal nerves will be given in connection with FIGS. 1A-1D. FIG. 1A depicts the upper cervical spine (C1-C4) area of an exemplary patient. As shown in FIG. 1A, a number of nerves arise from the upper cervical spine (C1-C4). Examples of such nerves include, but are not limited to, the greater occipital nerve(s) 101, lesser occipital nerve(s) 102, greater auricular nerve(s) 103, transverse cervical nerve(s) 104, supraclavicular nerve(s) 105, and/or branches of any of these nerves.

FIG. 1B depicts the occipital nerves 100 in the back of the head and upper neck area of a patient. As shown in FIG. 1B, the occipital nerves 100 are divided into greater and lesser occipital nerves 101 and 102, respectively, and are located on both sides of the midline. The greater occipital nerve 101 arises between the first and second cervical vertebrae and innervates the scalp at the top of the head, over the ear, and over the parotid glands. The lesser occipital nerve 102 also arises between the first and second cervical vertebrae and innervates the scalp in the lateral area of the head behind the ear.

As used herein, the term “occipital nerve” will be used to refer to one or more of the greater and lesser occipital nerves 101 and 102 on either or both sides of the midline. Because the occipital nerves 100 lie subcutaneously in the back of the head and upper neck, they are relatively easily accessed via surgical procedure.

FIGS. 1C and 1D depict the trigeminal nerve 106 and its branches. The trigeminal nerve 106 and its branches are responsible, in part, for the perception of head pain. The trigeminal nerve 106 on each side of the head arises from a trigeminal ganglion 107, which lies within the skull in an area known as Meckel's cave.

FIGS. 1C and 1D also show a number of branches of the trigeminal nerve 106. For example, the ophthalmic nerve 108, the maxillary nerve 109, the mandibular nerve 110, and the supraorbital nerve are all branches of the trigeminal nerve 106. As used herein, the term “trigeminal nerve” will be used to refer to the trigeminal nerve itself and/or one or more branches thereof on either or both sides of the midline.

As mentioned, medication overuse headache may be caused by or associated with the regular use of a number of different medications or drugs. One type of medication overuse headache is known as a “withdrawal” or “rebound” headache. A withdrawal headache may occur when a patient regularly takes medication to treat or prevent headaches and then tries to stop taking the medication. Additionally or alternatively, a withdrawal headache may occur when a patient regularly takes medication to treat or prevent a non-headache medical condition (e.g., non-headache pain) and then tries to stop taking the medication. As used herein, the term “medication overuse headache” will be used to refer to any type of headache caused by or otherwise associated with the regular use of any type of medication for any type of medical condition. For example, a medication overuse headache may be associated with one or more preventive, abortive, and/or rescue medications that are taken to treat headache.

Exemplary preventive medications that may be associated with medication overuse headache include, but are not limited to, tricyclic antidepressants (e.g., amitriptyline and nortriptyline), serotonin reuptake inhibitors (SSRIs) (e.g., paroxetine, venlafaxine, and fluoxetine), non-steroidal anti-inflammatory drugs (NSAIDs) (e.g., ibuprofen and ketoprofen), anticonvulsants (e.g. divalproex), and muscle relaxants.

Exemplary abortive medications that may be associated with medication overuse headache include, but are not limited to, barbiturates, triptans, acetaminophen, NSAIDS, aspirin, caffeine, ergotamine tartrates, opiods, and combination analgesics. Abortive medications are also referred to as acute medications.

Exemplary rescue medications that may be associated with medication overuse headache include, but are not limited to, butalbital compounds (e.g., Fiorinal and Fioricet), acetaminophens, oxycodone, hydrocodone (e.g., Vicodin, Percocet, and Tylenol), analgesics, injectable medications (e.g., Demerol), antinausea medications (e.g., Compazine, Phenergan, and Reglan), and muscle relaxants (e.g., Soma, Skelaxin, and Zanaflex).

It is believed that applying a stimulus to one or more stimulation sites within a patient may be useful in treating medication overuse headache. As used herein, the term “stimulation site” refers to one or more of the occipital nerves, one or more of the trigeminal nerves, and/or any other nerve or tissue associated with medication overuse headache. For example, stimulation of one or more of the occipital nerves and/or trigeminal nerves may be configured to relieve pain and/or any other symptom associated with medication overuse headache, thereby allowing the patient to decrease and/or eliminate the use of the medication(s) associated with the medication overuse headache.

Additionally or alternatively, the stimulation may be configured to gradually wean a medication overuse patient off of one or more medications associated with medication overuse headache. For example, continuous, intermittent, or bolus stimulation may be applied to one or more of the occipital nerves of a medication overuse patient over a period of time. The stimulation may be adjusted or altered over time to allow the patient to slowly wean himself or herself from one or more medications associated with medication overuse headache.

In some alternative examples, the stimulation may be configured to treat medication overuse headaches associated with one or more medications being used by a patient to treat a non-headache medical condition. For example, a patient may regularly take one or more medications such as, but not limited to, NSAIDs, for non-headache pain. Once the pain subsides, the patient may desire to cease taking the medications. In so doing, the patient may experience medication overuse headache (e.g., withdrawal headache). In some instances, the patient may revert back to taking the medications in order to avoid the withdrawal headaches. Hence, in some examples, stimulation may be applied to one or more stimulation sites within the patient in accordance with the systems and methods described herein in order to minimize the effects of the withdrawal headaches as the patient attempts to decrease and/or eliminate the intake of medications.

Consequently, a stimulator may be implanted within a patient to deliver a stimulus to one or more stimulation sites within the patient to treat medication overuse headache. The stimulus may include an electrical stimulation current, one or more drugs or other chemical stimulation, thermal stimulation, electromagnetic stimulation, mechanical stimulation, and/or any other suitable stimulation.

As used herein, and in the appended claims, the term “stimulator” will be used broadly to refer to any device that delivers a stimulus to a stimulation site (e.g., one or more of the occipital nerves and/or trigeminal nerves) to treat medication overuse headache. Thus, the term “stimulator” includes, but is not limited to, a microstimulator, implantable pulse generator (IPG), spinal cord stimulator (SCS), external trial stimulator, system control unit, deep brain stimulator, drug pump, or similar device.

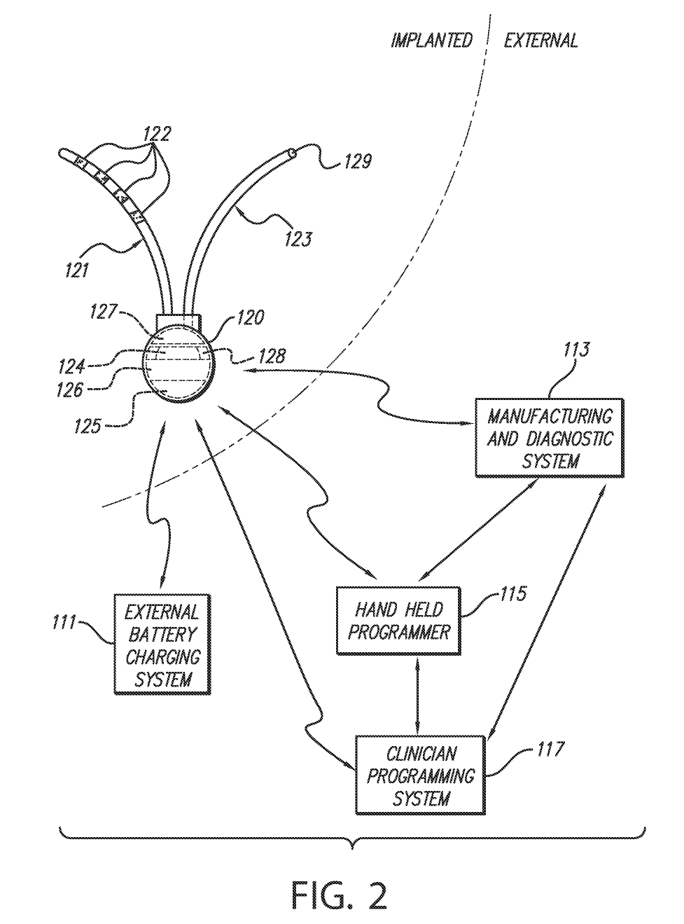

A more detailed description of an exemplary stimulator and its operation will now be given in connection with FIG. 2. FIG. 2 illustrates an exemplary stimulator 120 that may be used to apply a stimulus to a stimulation site within a patient, e.g., an electrical stimulation of the stimulation site, an infusion of one or more drugs at the stimulation site, or both. The electrical stimulation function of the stimulator 120 will be described first, followed by an explanation of the possible drug delivery function of the stimulator 120. It will be understood, however, that the stimulator 120 may be configured to provide only electrical stimulation, only drug stimulation, both types of stimulation, or any other type of stimulation as best suits a particular patient.

The exemplary stimulator 120 shown in FIG. 2 is configured to provide electrical stimulation to one or more stimulation sites within a patient and may include at least one lead 121 coupled thereto. In some examples, the at least one lead 121 includes a number of electrodes 122 through which electrical stimulation current may be applied to a stimulation site. It will be recognized that the at least one lead 121 may include any number of electrodes 122 arranged in any configuration as best serves a particular application. In some alternative examples, as will be described in more detail below, the stimulator 120 is leadless.

As illustrated in FIG. 2, the stimulator 120 includes a number of components. It will be recognized that the stimulator 120 may include additional and/or alternative components as best serves a particular application. A power source 125 is configured to output voltage used to supply the various components within the stimulator 120 with power and/or to generate the power used for electrical stimulation. The power source 125 may include a primary battery, a rechargeable battery (e.g., a lithium-ion battery), a super capacitor, a nuclear battery, a mechanical resonator, an infrared collector (receiving, e.g., infrared energy through the skin), a thermally-powered energy source (where, e.g., memory-shaped alloys exposed to a minimal temperature difference generate power), a flexural powered energy source (where a flexible section subject to flexural forces is part of the stimulator), a bioenergy power source (where a chemical reaction provides an energy source), a fuel cell, a bioelectrical cell (where two or more electrodes use tissue-generated potentials and currents to capture energy and convert it to useable power), an osmotic pressure pump (where mechanical energy is generated due to fluid ingress), or the like.

In some examples, the power source 125 may be recharged using an external charging system. One type of rechargeable power supply that may be used is described in U.S. Pat. No. 6,596,439, which is incorporated herein by reference in its entirety. Other battery construction techniques that may be used to make the power source 125 include those shown, e.g., in U.S. Pat. Nos. 6,280,873; 6,458,171; 6,605,383; and 6,607,843, all of which are incorporated herein by reference in their respective entireties.

The stimulator 120 may also include a coil 128 configured to receive and/or emit a magnetic field (also referred to as a radio frequency (RF) field) that is used to communicate with, or receive power from, one or more external devices. Such communication and/or power transfer may include, but is not limited to, transcutaneously receiving data from the external device, transmitting data to the external device, and/or receiving power used to recharge the power source 125.

For example, an external battery charging system (EBCS) 111 may be provided to generate power that is used to recharge the power source 125 via any suitable communication link. Additional external devices including, but not limited to, a hand held programmer (HHP) 115, a clinician programming system (CPS) 117, and/or a manufacturing and diagnostic system (MDS) 113 may also be provided and configured to activate, deactivate, program, and/or test the stimulator 120 via one or more communication links. It will be recognized that the communication links shown in FIG. 2 may each include any type of link used to transmit data or energy, such as, but not limited to, an RF link, an infrared (IR) link, an optical link, a thermal link, or any other energy-coupling link.

Additionally, if multiple external devices are used in the treatment of a patient, there may be communication among those external devices, as well as with the implanted stimulator 120. It will be recognized that any suitable communication link may be used among the various devices illustrated.

The external devices shown in FIG. 2 are merely illustrative of the many different external devices that may be used in connection with the stimulator 120. Furthermore, it will be recognized that the functions performed by any two or more of the external devices shown in FIG. 2 may be performed by a single external device.

The stimulator 120 may also include electrical circuitry 124 configured to generate the electrical stimulation current that is delivered to a stimulation site via one or more of the electrodes 122. For example, the electrical circuitry 124 may include one or more processors, capacitors, integrated circuits, resistors, coils, and/or any other component configured to generate electrical stimulation current.

Additionally, the exemplary stimulator 120 shown in FIG. 2 may be configured to provide drug stimulation to a patient by applying one or more drugs at a stimulation site within the patient. To this end, a pump 127 may also be included within the stimulator 120. The pump 127 is configured to store and dispense one or more drugs, for example, through a catheter 123. The catheter 123 is coupled at a proximal end to the stimulator 120 and may have an infusion outlet 129 for infusing dosages of the one or more drugs at the stimulation site. In some embodiments, the stimulator 120 may include multiple catheters 123 and/or pumps 127 for storing and infusing dosages of the one or more drugs at the stimulation site.

The stimulator 120 may also include a programmable memory unit 126 configured to store one or more stimulation parameters. The stimulation parameters may include, but are not limited to, electrical stimulation parameters, drug stimulation parameters, and other types of stimulation parameters. The programmable memory unit 126 allows a patient, clinician, or other user of the stimulator 120 to adjust the stimulation parameters such that the stimulation applied by the stimulator 120 is safe and efficacious for treatment of a particular patient. The programmable memory unit 126 may include any type of memory unit such as, but not limited to, random access memory (RAM), static RAM (SRAM), a hard drive, or the like.

The electrical stimulation parameters may control various parameters of the stimulation current applied to a stimulation site including, but not limited to, the frequency, pulse width, amplitude, waveform (e.g., square or sinusoidal), electrode configuration (i.e., anode-cathode assignment), burst pattern (e.g., continuous or intermittent), duty cycle or burst repeat interval, ramp on time, and ramp off time. The drug stimulation parameters may control various parameters including, but not limited to, the amount of drugs infused at the stimulation site, the rate of drug infusion, and the frequency of drug infusion. For example, the drug stimulation parameters may cause the drug infusion rate to be intermittent, continuous, or bolus. Other stimulation parameters that characterize other classes of stimuli are possible. For example, when tissue is stimulated using electromagnetic radiation, the stimulation parameters may characterize the intensity, wavelength, and timing of the electromagnetic radiation stimuli. When tissue is stimulated using mechanical stimuli, the stimulation parameters may characterize the pressure, displacement, frequency, and timing of the mechanical stimuli.

Specific stimulation parameters may have different effects on different types, causes, or symptoms of medication overuse headache. Thus, in some examples, the stimulation parameters may be adjusted at any time throughout the treatment course as best serves the particular patient being treated. It will be recognized that any of the characteristics of the stimulation current, including, but not limited to, the pulse shape, amplitude, pulse width, frequency, burst pattern (e.g., continuous, cycled, or intermittent), duty cycle or burst repeat interval, ramp on time, and ramp off time may be adjusted as best serves a particular application.

To illustrate, a baseline set of stimulation parameters may initially be set to begin treatment of medication overuse headache. These baseline values may be adjusted throughout the course of treatment in response to patient feedback or sensed indicators of medication overuse headache. Additionally or alternatively, the patient and/or clinician may adjust the stimulation parameters at any time to prevent accommodation, collateral stimulation, and/or ineffectiveness.

An exemplary baseline set of stimulation parameters that may be used to initially define stimulation current that is used to treat medication overuse headache includes, but is not limited to the stimulation parameters shown in Table 1. It will be recognized that the baseline set of stimulation parameters shown in Table 1 may vary depending on the particular patient being treated and that additional or alternative stimulation parameters may be defined.

TABLE 1
Exemplary Baseline Stimulation Parameters

| | |
|---|---|
| Pulse width | 250 microseconds (μsec) |
| Frequency | 60 Hertz (Hz) |
| Burst pattern | Continuous |
| Amplitude | 0.2-20 milliamps (mA) |

Hence, as shown in Table 1, a continuous stimulation current having a pulse width of 250 μsec, a frequency of 60 Hz, and an amplitude anywhere between 0.2 to 20 mA may be initially applied to one or more stimulation sites of a patient (e.g., one or more of the occipital nerves and/or trigeminal nerves) in order to treat medication overuse headache.

After a predetermined length of time (e.g., a week, a month, or multiple months) of treatment, the patient may be evaluated to determine whether additional stimulation is needed in order to treat medication overuse headache. In some examples, if the patient has successfully been weaned from the medications associated with medication overuse headache and if the patient no longer experiences the symptoms of headache, the stimulation may be terminated. Alternatively, if it is determined that the patient needs further treatment, the stimulation may continue in accordance with the same set of stimulation parameters or in accordance with a newly defined set of stimulation parameters. For example, the stimulation parameters may be adjusted from the exemplary baseline stimulation parameters described previously in connection with Table 1 to have the exemplary values within the ranges shown in Table 2:

TABLE 2
Exemplary Adjusted Stimulation Parameters

| Pulse width | 50-1000 | μsec |
|---|---|---|
| Frequency | 2-1200 | Hz |
| Burst pattern | Cycled | |
| Amplitude | 0-20 | mA |

As shown in Table 2, the pulse width, frequency, and/or amplitude may be adjusted so that the stimulation current more effectively treats medication overuse headache. For example, the pulse width may be adjusted to a suitable value in between and including 50 and 1000 μsec, the frequency may be adjusted to a suitable value in between and including 2 and 1200 Hz, the burst pattern may be adjusted to a cycled pattern, and/or the amplitude may be adjusted to a suitable value in between 0 and 20 mA. It will be recognized that the values shown in Table 2 are merely illustrative and that they may vary as may serve a particular application. It will also be recognized that any other stimulation parameter may be adjusted in order to more effectively treat medication overuse headache.

The stimulator 120 of FIG. 2 is illustrative of many types of stimulators that may be used in accordance with the systems and methods described herein. For example, the stimulator 120 may include an implantable pulse generator (IPG), a spinal cord stimulator (SCS), a deep brain stimulator, a drug pump, or any other type of implantable device configured to deliver a stimulus to a stimulation site within a patient. Exemplary IPGs suitable for use as described herein include, but are not limited to, those disclosed in U.S. Pat. Nos. 6,381,496, 6,553,263; and 6,760,626. Exemplary spinal cord stimulators suitable for use as described herein include, but are not limited to, those disclosed in U.S. Pat. Nos. 5,501,703; 6,487,446; and 6,516,227. Exemplary deep brain stimulators suitable for use as described herein include, but are not limited to, those disclosed in U.S. Pat. Nos. 5,938,688; 6,016,449; and 6,539,263. All of these listed patents are incorporated herein by reference in their respective entireties.

The stimulator 120 of FIG. 2 may alternatively include a microstimulator. Various details associated with the manufacture, operation, and use of implantable microstimulators are disclosed in U.S. Pat. Nos. 5,193,539; 5,193,540; 5,312,439; 6,185,452; 6,164,284; 6,208,894; and 6,051,017. All of these listed patents are incorporated herein by reference in their respective entireties.

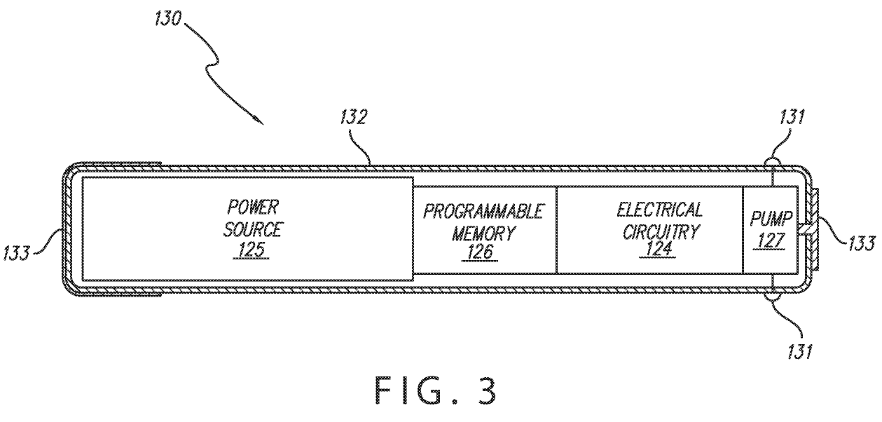

FIG. 3 illustrates an exemplary microstimulator 130 that may be used as the stimulator 120 described herein. Other configurations of the microstimulator 130 are possible, as shown in the above-referenced patents and as described further below.

As shown in FIG. 3, the microstimulator 130 may include the power source 125, the programmable memory 126, the electrical circuitry 124, and the pump 127 described in connection with FIG. 2. These components are housed within a capsule 132. The capsule 132 may be a thin, elongated cylinder or any other shape as best serves a particular application. The shape of the capsule 132 may be determined by the structure of the desired stimulation site and the method of implantation. In some examples, the microstimulator 130 may include two or more leadless electrodes 133 disposed on the outer surface thereof.

The external surfaces of the microstimulator 130 may advantageously be composed of biocompatible materials. For example, the capsule 132 may be made of glass, ceramic, metal, or any other material that provides a hermetic package that will exclude water vapor but permit passage of electromagnetic fields used to transmit data and/or power. The electrodes 133 may be made of a noble or refractory metal or compound, such as platinum, iridium, tantalum, titanium, titanium nitride, niobium or alloys of any of these, in order to avoid corrosion or electrolysis which could damage the surrounding tissues and the device.

The microstimulator 130 may also include one or more infusion outlets 131 configured to dispense one or more drugs directly at a stimulation site. Alternatively, one or more catheters may be coupled to the infusion outlets 131 to deliver the drug therapy to a treatment site some distance from the body of the microstimulator 130.

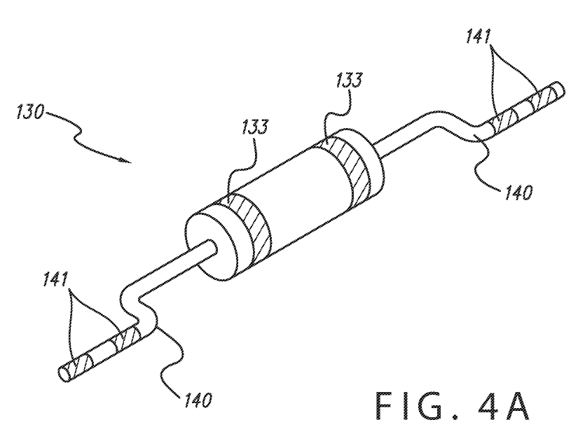

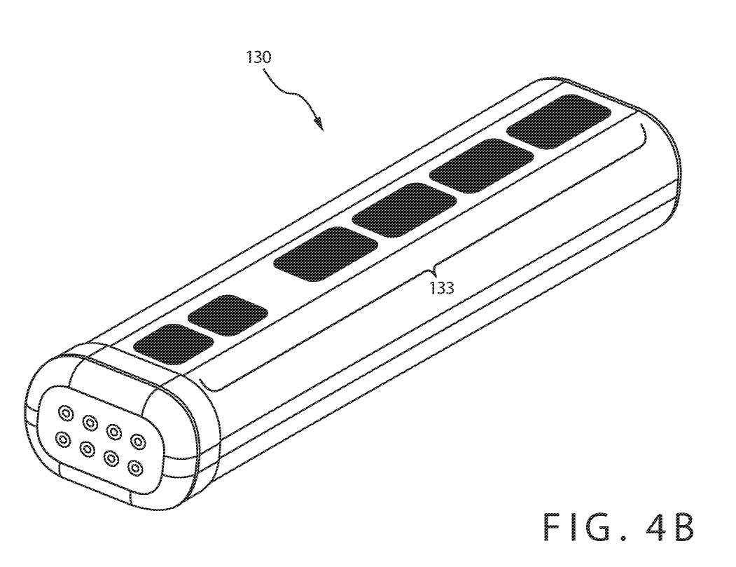

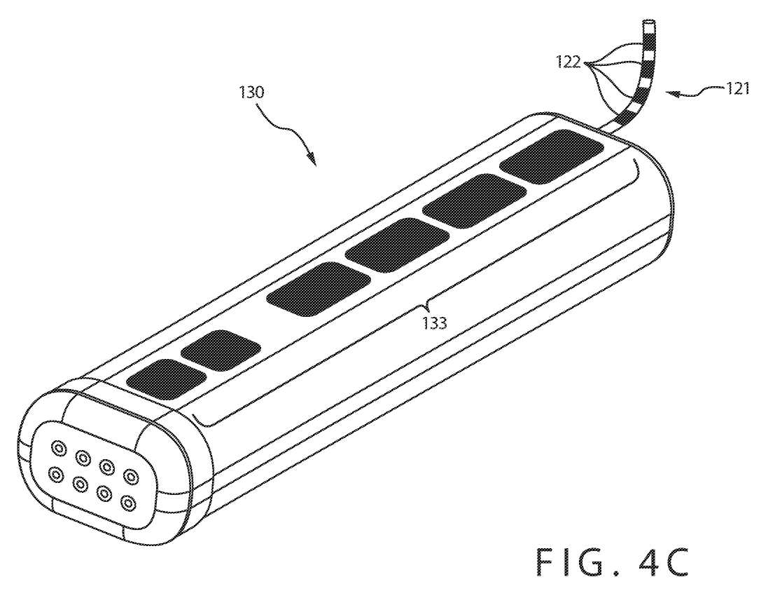

FIGS. 4A-4C show alternative configurations of a microstimulator 130. It will be recognized that the alternative configurations shown in FIGS. 4A-4C are merely illustrative of the many possible configurations of a microstimulator 130. For example, FIG. 4A shows an example of a microstimulator 130 with one or more leads 140 coupled thereto. As shown in FIG. 4A, each of the leads 140 may include one or more electrodes 141 disposed thereon. The microstimulator 130 of FIG. 4A may additionally or alternatively include one or more leadless electrodes 133 disposed on the outer surface thereof.

FIG. 4B illustrates an exemplary microstimulator 130 with a plurality of electrodes 133 disposed on an outer surface thereof. In some examples, any number of electrodes 133 may be disposed on the outer surface of the microstimulator 130. In some alternative examples, as shown in FIG. 4C, the microstimulator 130 may be coupled to a lead 121 having a number of electrodes 122 disposed thereon. Each of the electrodes 133 and 122 may be selectively configured to serve as an anode or as a cathode.

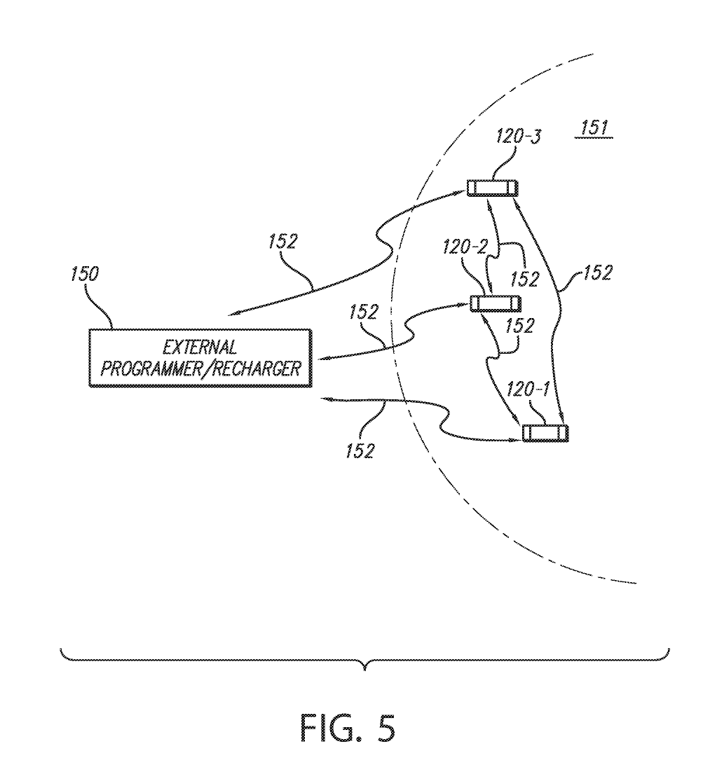

In some examples, the stimulator 120 of FIG. 2 may be configured to operate independently. Alternatively, as shown in FIG. 5, the stimulator 120 may be configured to operate in a coordinated manner with one or more additional stimulators, other implanted devices, or other devices external to the patient's body. FIG. 5 illustrates an exemplary configuration wherein a first stimulator 120-1 implanted within the patient 151 provides a stimulus to a first location, a second stimulator 120-2 provides a stimulus to a second location, and a third stimulator 120-3 provides a stimulus to a third location. In some examples, one or more external devices 150 may be configured to control the operation of each of the implanted devices 120. In some embodiments, an implanted device, e.g., stimulator 120-1, may control, or operate under the control of, another implanted device(s), e.g., stimulator 120-2 and/or stimulator 120-3. Control lines 152 have been drawn in FIG. 5 to illustrate that the external device 150 may communicate or provide power to any of the implanted devices 120 and that each of the various implanted devices 120 may communicate with and, in some instances, control any of the other implanted devices.

As a further example of multiple stimulators 120 operating in a coordinated manner, the first and second stimulators 120-1 and 120-2 of FIG. 5 may be configured to sense various indicators of the symptoms or causes of medication overuse headache and transmit the measured information to the third stimulator 120-3. The third stimulator 120-3 may then use the measured information to adjust its stimulation parameters and apply stimulation to a stimulation site accordingly. The various implanted stimulators may, in any combination, sense indicators of medication overuse headache, communicate or receive data regarding such indicators, and adjust stimulation parameters accordingly.

In order to determine the strength and/or duration of electrical stimulation and/or amount and/or type(s) of stimulating drug(s) required to most effectively treat medication overuse headache, various indicators of medication overuse headache and/or a patient's response to treatment may be sensed or measured. The stimulator 120 may then adjust the stimulation parameters (e.g., in a closed loop manner) in response to one or more of the sensed indicators. Exemplary indicators include, but are not limited to, electrical activity of the brain (e.g., EEG), neurotransmitter levels, hormone levels, neuropeptide levels (e.g., substance P levels and calcitonin gene-related peptide (CGRP) levels), metabolic activity in the brain, blood flow rate, medication levels within the patient, patient input, temperature of the stimulation site, physical activity level, brain hyperexcitability, and/or indicators of collateral tissue stimulation. In some examples, the stimulator 120 may be configured to perform the measurements. Alternatively, other sensing devices may be configured to perform the measurements and transmit the measured values to the stimulator 120. Exemplary sensing devices include, but are not limited to, chemical sensors, electrodes, optical sensors, mechanical (e.g., motion, pressure) sensors, and temperature sensors.

Thus, one or more external devices may be provided to interact with the stimulator 120, and may be used to accomplish at least one or more of the following functions:

Function 1: If necessary, transmit electrical power to the stimulator 120 in order to power the stimulator 120 and/or recharge the power source 125.

Function 2: Transmit data to the stimulator 120 in order to change the stimulation parameters used by the stimulator 120.

Function 3: Receive data indicating the state of the stimulator 120 (e.g., battery level, drug level, stimulation parameters, etc.).

Additional functions may include adjusting the stimulation parameters based on information sensed by the stimulator 120 or by other sensing devices.

By way of example, an exemplary method of treating medication overuse headache may be carried out according to the following sequence of procedures. The steps listed below may be modified, reordered, and/or added to as best serves a particular application.

1. A stimulator 120 is implanted so that its electrodes 122 and/or infusion outlet 129 are in communication with a stimulation site within a patient. As used herein and in the appended claims, the term “in communication with” refers to the stimulator 120, stimulating electrodes 122, and/or infusion outlet 129 being adjacent to, in the general vicinity of, in close proximity to, directly next to, or directly on the stimulation site.

2. The stimulator 120 is programmed to apply at least one stimulus to the stimulation site. The stimulus may include electrical stimulation, drug stimulation, gene infusion, chemical stimulation, thermal stimulation, electromagnetic stimulation, mechanical stimulation, and/or any other suitable stimulation.

3. When the patient desires to invoke stimulation, the patient sends a command to the stimulator 120 (e.g., via a remote control) such that the stimulator 120 delivers the prescribed stimulation to the stimulation site. For example, the stimulation may be activated by the patient when the patient feels the onset of a headache. The stimulator 120 may alternatively or additionally be configured to apply the stimulation to the stimulation site in accordance with one or more pre-determined stimulation parameters and/or automatically apply the stimulation in response to sensed indicators of medication overuse headache.

4. To cease stimulation, the patient may turn off the stimulator 120 (e.g., via a remote control).

5. Periodically, the power source 125 of the stimulator 120 is recharged, if necessary, in accordance with Function 1 described above.

In other examples, the treatment administered by the stimulator 120, i.e., drug therapy and/or electrical stimulation, may be automatic and not controlled or invoked by the patient. It will be recognized that the particular stimulation methods and parameters may vary as best serves a particular application.

The stimulator 120 may be implanted within a patient using any suitable surgical procedure such as, but not limited to, small incision, open placement, laparoscopy, or endoscopy. Exemplary methods of implanting a microstimulator, for example, are described in U.S. Pat. Nos. 5,193,539; 5,193,540; 5,312,439; 6,185,452; 6,164,284; 6,208,894; and 6,051,017. Exemplary methods of implanting an SCS, for example, are described in U.S. Pat. Nos. 5,501,703; 6,487,446; and 6,516,227. Exemplary methods of implanting a deep brain stimulator, for example, are described in U.S. Pat. Nos. 5,938,688; 6,016,449; and 6,539,263. All of these listed patents are incorporated herein by reference in their respective entireties.

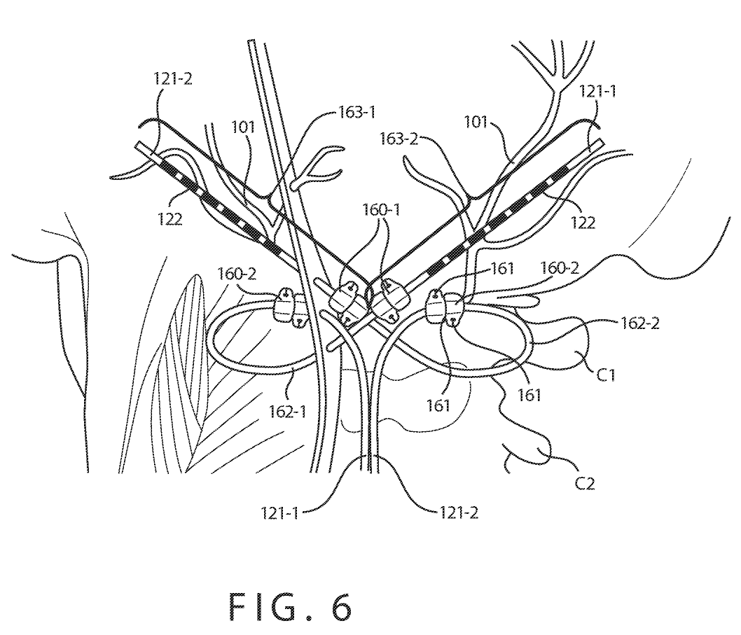

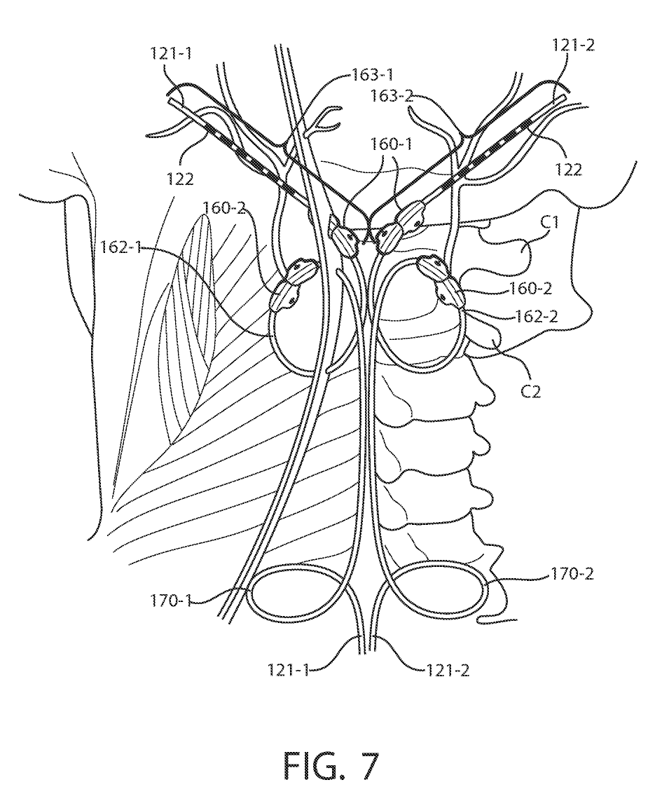

To illustrate, FIGS. 6-7 illustrate exemplary configurations wherein one or more electrodes 122 coupled to an implantable stimulator 120 are in communication with one or more of the occipital nerves 100. The configurations shown in FIGS. 6-7 are more fully described in co-pending and commonly-assigned U.S. patent application Ser. No. 11/728,816 to Jaax et al. and entitled “METHODS AND SYSTEMS FOR FACILITATING STIMULATION OF ONE OR MORE STIMULATION SITES,” the contents of which are incorporated herein by reference in their entirety.

In the example of FIG. 6, the electrodes 122 are disposed on a distal portion 163 of one or more leads 121 that are coupled to a stimulator 120. The number of leads 121 and the number of electrodes 122 disposed on each lead 121 may vary as may serve a particular application.

As shown in FIG. 6, the lead configuration may include two leads (e.g., 121-1 and 121-2, collectively referred to herein as 121). In this example, a distal portion 163-1 of the first lead 121-1 is positioned over the greater occipital nerve 101-1 on the right side of the patient and a distal portion 163-2 of the second lead 121-2 is placed over the greater occipital nerve 101-2 on the left side of the patient. The distal portions 163 of the leads 121 shown in FIG. 6 and in the other examples described herein are straight for illustrative purposes only. It will be recognized that the distal portions 163 may alternatively be curved, helical, paddle-shaped, or of any other shape as may serve a particular application.

The distal portions 163 of each of the leads 121 shown in FIG. 6 cover the greater occipital nerves 101 for illustrative purposes only. It will be recognized that the leads 121 may be located at any other stimulation site (e.g., the lesser occipital nerve 102) as may serve a particular application. In some examples, the distal tip of each of the leads 121 is placed four to five centimeters from the midline (i.e., the medial line or plane of the body) to minimize the need to advance the leads 121 following insertion. However, it will be recognized that the leads 121 may be placed any distance from the midline.

Configurations having two leads 121, such as that shown in FIG. 6, are advantageous in applications wherein it is desirable to apply stimulation to multiple stimulation sites. For example, the lead configuration of FIG. 6 may be used to simultaneously apply stimulation to locations on both the right and left sides of the patient. However, it will be recognized that a single lead 121 or more than two leads 121 may be used in accordance with the systems and methods described herein.

Each lead 121 is secured by one or more suture sleeves 160—e.g., a distal suture sleeve 160-1 and a proximal suture sleeve 160-2. The proximal suture sleeve 160-2 is closer to the stimulator (not shown) than is the distal suture sleeves 160-1. It will be recognized that any number of suture sleeves 160 may be used to secure the leads 121 in place. Moreover, it will be recognized that any other securing device may additionally or alternatively be used to secure the leads 121 in place. Such securing devices may include, but are not limited to, one or more sutures, hooks, adhesives, or anchors.

Each suture sleeve 160 may be sutured into place using one or more sutures 161. In some examples, the sutures are non-absorbable. Exemplary non-absorbable sutures that may be used to suture the suture sleeves 160 into place include, but are not limited to, a braided nylon (e.g., Nurolon), a braided polyester (e.g., Ethibond or Mersiline), Prolene, Surgilene, Tevdek, a polypropylene material, a braided polyester material, and a Teflon coated polyester material.

As shown in FIG. 6, the long axis of each distal suture sleeve 160-1 is substantially collinear with the long axis of the electrode region of its corresponding lead 121. Each lead 121 passes through the lumen 171 of its corresponding distal suture sleeve 160-1 and then forms a loop (e.g., 162-1 and 162-2, collectively referred to herein as 162) of at least 360 degrees. To this end, the leads 121 are configured to pass through corresponding proximal sleeves 160-2, which are positioned so as to maintain the shape of the loops 162. The leads 121 may then be routed to the stimulator (not shown). The portion of the leads 121 that makes up each loop 162 may be made out of any flexible material.

The loops 162 are configured to minimize the forces that are exerted on the distal and proximal sutures sleeves 160-1 and 160-2 when the patient moves his or her head. Hence, the loops 162 are also referred to as “force redirection loops” herein. The force redirection loops 162 are also configured to minimize lead migration. Hence, the force redirection loops 162 may be dimensioned and aligned such that there are minimal forces on either the distal or proximal suture sleeve 160-1 or 160-2.

In some examples, each lead 121 crosses the midline prior to forming its corresponding force redirection loop 162. For example, as shown in FIG. 6, the electrode portion of lead 121-1 is located on the right side of the midline. The lead 121-1 crosses the midline prior to forming force redirection loop 162-1. Lead 121-2 also crosses the midline prior to forming force redirection loop 162-2. Hence, the leads 121 cross each other prior to forming force redirection loops 162. Alternatively, as will be described in more detail below, the leads 121 may be positioned such that they form force redirection loops 162 without crossing each other.

As mentioned, each lead 121 passes through a corresponding proximal suture sleeve 160-2 in forming a force redirection loop 162. As shown in FIG. 6, the long axis of each proximal suture sleeve 160-2 may be substantially perpendicular to the midline or spine. This placement minimizes lead migration that may be caused by the flexion or extension of the neck. Such flexion or extension of the neck may cause the proximal suture sleeve 160-2 to bend, however, the risk of the lead 121 slipping within the suture sleeve 160-2 is minimized when the proximal suture sleeve 160-2 is perpendicular to the midline or spine. Alternatively, as will be described in more detail below, the long axis of the proximal suture sleeve 160-2 may be oriented in any non-parallel direction with respect to the midline.

After the leads 121 pass through corresponding proximal suture sleeves 160-2, the leads 121 are routed to a stimulator 120. In some examples, as will be described in more detail below, the leads 121 may form one or more additional loops prior to being coupled to the stimulator 120. It will be recognized that one or more devices, such as the stimulator 120, may be implanted in any suitable location within the body. For example, the stimulator 120 may be implanted above the iliac crest or over the ribcage to minimize the path of the leads 121 and to minimize the need for multiple lead extensions. Other exemplary implant locations may include, but are not limited to, the buttocks, neck, brain, and subcutaneous area on top of the skull, or any other suitable location within the patient.

FIG. 7 illustrates an alternative lead configuration that may be used in connection with the systems and methods described herein. The lead configuration of FIG. 7 is similar to that described in connection with FIG. 6 in that the configuration includes multiple leads 121 and suture sleeves 160 configured to secure the leads 121 in place. The leads 121 may each include one or more electrodes 122 disposed thereon and are implanted such that one or more of the electrodes 122 are in communication with one or more stimulation sites (e.g., the greater occipital nerves 101).

As shown in FIG. 7, each distal suture sleeve 160-1 may be substantially collinear with the long axis of the distal portion 163 of its corresponding lead 121. Each distal suture sleeve 160-1 is sutured or otherwise fixed to fascia or any other securing site that is located, for example, in the same vertebral level as the most proximal electrode 122 on the lead 121 to minimize relative movement between the target stimulation site (e.g., the greater occipital nerve 101) and the distal suture sleeve 160-1. For example, if the most proximate electrode 122 to the distal suture sleeve 160-1 is located in the C2 region, the distal suture sleeve 160-1 is sutured to fascia in the same C2 region. Likewise, if the most proximate electrode 122 to the distal suture sleeve 160-1 is located in the scalp region, the distal suture sleeve 160-1 is sutured to fascia overlying the scalp.

As described previously in connection with the example of FIG. 6, each lead 121 passes through the lumen 171 of its corresponding distal suture sleeve 160-1 and then forms a force redirection loop (e.g., 162-1 and 162-2) of at least 360 degrees before passing through the proximal sleeves 160-2. However, as shown in the example of FIG. 7, each lead 121 forms the force redirection loop 162 without crossing the other lead 121. For example, lead 121-1 forms force redirection loop 162-1 without crossing lead 121-2. In some examples, each lead 121 forms a force redirection loop 162 without crossing the midline.

After passing through the proximal suture sleeves 160-2, the leads 121 may each be formed into one or more additional loops (e.g., 170-1 and 170-2, collectively referred to herein as 170) prior to being routed to the stimulator 120. These additional loops 170 may relieve strain that may be placed on the leads 121 by changing size as the patient moves. Hence, these additional loops 170 are referred to herein as “strain relief loops” for illustrative purposes.

FIG. 7 shows that the strain relief loops 170 may be formed at the base of the neck. Additionally or alternatively, the strain relief loops 170 may be formed at any other suitable location as may serve a particular application.

In some examples, the strain relief loops 170 are located within a pocket made by a surgeon in the subcutaneous fat and are not sutured or otherwise affixed to tissue. In this manner, the fat retains the general shape of the strain relief loops 170 while allowing the loops 170 to vary in size as the patient moves.

Additionally or alternatively, the leads 121 may each form a strain relief loop at or near the location of the stimulator 120. For example, if the stimulator 120 is implanted over the ribcage, lead(s) 121 may form one or more strain relief loops at or near the rib cage just prior to being coupled to the stimulator 120.

It will be recognized that the implant configurations shown in FIGS. 6-7 are merely illustrative and that the stimulator 120, leads 121, and/or electrodes 122 may additionally or alternatively be implanted using any other suitable technique as may serve a particular application. Exemplary devices, implant techniques, and stimulation configurations that may be used in connection with the systems and methods described herein are described in U.S. Pat. No. 6,735,475; U.S. Patent Application Publication Nos. 20050102006, 20060293723, 20060206165, and 20060064140; and co-pending and commonly-assigned U.S. patent application Ser. Nos. 11/280,620, entitled “IMPLANTABLE STIMULATOR,” filed Nov. 16, 2005, and 11/280,582, entitled “ELECTRODE CONTACT CONFIGURATIONS FOR AN IMPLANTABLE STIMULATOR,” filed Nov. 16, 2005. Each of these patents and patent applications are incorporated herein by reference in their respective entireties.

For instance, as shown in FIGS. 5A-5B of U.S. Patent Application Publication No. 20060064140, a microstimulator having a plurality of electrodes disposed thereon may be implanted within a patient such that the microstimulator is in communication with a stimulation site within the patient (e.g., the occipital and/or trigeminal nerve). Alternatively, a microstimulator coupled to a lead 121 having a number of electrodes 122 disposed thereon may be implanted within a patient such that the electrodes 122 are in communication with a stimulation site within the patient (e.g., the occipital and/or trigeminal nerve).

The preceding description has been presented only to illustrate and describe embodiments of the invention. It is not intended to be exhaustive or to limit the invention to any precise form disclosed. Many modifications and variations are possible in light of the above teaching.

What is claimed is:

1. A method of treating a patient with medication overuse headache, comprising:
providing a stimulator;
configuring one or more stimulation parameters to treat medication overuse headache;
programming said stimulator with said one or more stimulation parameters;
generating a stimulus configured to treat said medication overuse headache with said stimulator in accordance with said one or more stimulation parameters; and
applying said stimulus with said stimulator to a stimulation site within said patient.

2. The method of claim 1, wherein said stimulation site comprises at least one or more of an occipital nerve and a trigeminal nerve.

3. The method of claim 1, wherein said medication overuse headache is associated with at least one or more of a preventive medication, an abortive medication, and a rescue medication.

4. The method of claim 1, wherein said stimulator is coupled to one or more electrodes, and wherein said stimulus comprises a stimulation current delivered via said electrodes.

5. The method of claim 1, further comprising evaluating an effectiveness of said stimulus and adjusting said stimulation parameters in accordance with said evaluation.

6. The method of claim 1, further comprising at least partially implanting said stimulator within said patient.

7. The method of claim 1, wherein said stimulus is configured to wean said patient off of one or more medications.

8. The method of claim 1, further comprising sensing at least one indicator related to said medication overuse headache and using said at least one sensed indicator to adjust one or more of said stimulation parameters.

9. A method of treating medication overuse headache, said method comprising:
implanting a stimulator at least partially within a patient;
configuring one or more stimulation parameters to treat medication overuse headache;
programming said stimulator with said one or more stimulation parameters;
generating a stimulation current configured to treat said medication overuse headache with said stimulator in accordance with said one or more stimulation parameters; and
applying said stimulation current with said implanted stimulator to a stimulation site within said patient.

10. The method of claim 9, wherein said stimulation site comprises at least one or more of an occipital nerve and a trigeminal nerve.

11. The method of claim 9, wherein said medication overuse headache is associated with at least one or more of a preventive medication, an abortive medication, and a rescue medication.

12. The method of claim 9, further comprising evaluating an effectiveness of said stimulus and adjusting said stimulation parameters in accordance with said evaluation.

13. The method of claim 9, further comprising:
coupling at least one lead having at least one electrode disposed thereon to said stimulator;
implanting said at least one lead within said patient such that said at least one electrode is in communication with said stimulation site; and
applying said stimulation current via said at least one electrode to said stimulation site.

14. The method of claim 9, wherein said stimulation current is configured to wean said patient off of one or more medications.

15. The method of claim 9, further comprising sensing at least one indicator related to said medication overuse headache and using said at least one sensed indicator to adjust one or more of said stimulation parameters.

16. A system for treating a patient with medication overuse headache, said system comprising:
a stimulator configured to generate at least one stimulus in accordance with one or more stimulation parameters adjusted to treat medication overuse headache;
a programmable memory unit in communication with said stimulator and programmed to store said one or more stimulation parameters to at least partially define said stimulus such that said stimulus is configured to treat said medication overuse headache; and
means, operably connected to said stimulator, for applying said stimulus to a stimulation site within said patient.

17. The system of claim 16, wherein said stimulation site comprises at least one or more of an occipital nerve and a trigeminal nerve.

18. The system of claim 16, wherein said medication overuse headache is associated with at least one or more of a preventive medication, an abortive medication, and a rescue medication.

19. The system of claim 16, wherein said means for applying said at least one stimulus comprises one or more electrodes, and wherein said stimulus comprises a stimulation current delivered via said electrodes.

20. The system of claim 16, further comprising:
a sensor configured to sense at least at least one indicator related to said medication overuse headache;
wherein said stimulator is further configured to adjust said stimulation parameters in accordance with said sensed indicator.
